(19) United States
(12) Patent Application Publication
(10) Pub. No.: US 2007/0053981 A1
(43) Pub. Date: Mar. 8, 2007

(54) Solid composition comprising a proton pump inhibitor

(76) Inventors: Eva Blychert, Molndal (SE); Marjo Janssen, Zoetermeer (NL)

Correspondence Address: WHITE & CASE LLP;PATENT DEPARTMENT, 1155 AVENUE OF THE AMERICAS, NEW YORK, NY, 10036, US

(21) Appl. No.: 10/564,229

(22) PCT Filed: Jul. 8, 2004
(86) PCT No.: PCT/SE04/01113

§ 371 Date: Oct. 2, 2006

Related U.S. Application Data
(60) Provisional application No. 60/486,795, filed on Jul. 11, 2003.

(51) Int. Cl. A61K 9/26

(52) U.S. Cl. 424/470

(57) ABSTRACT

The present invention related to a method for oral administration of a solid composition comprising an acid labile proton pump inhibitor compound in the form of a multiple of enteric coating layered pellets, wherein the pellets are in admixture with one or more pharmaceutically acceptable thickeners capable of forming a viscous medium when dispersed in an aqueous carrier. Alternatively, the enteric coated pellets are in admixture with a viscous medium. The formed aqueous viscous suspension is administered through a gastric tube. The method and composition are especially aimed for treatment of patients in need of a proton pump inhibitor and having difficulties to swallow or for pediatric patients.

### FIELD OF THE INVENTION

The present invention relates to a method for oral administration of an acid labile heterocyclic compound with gastric acid inhibitory effect, in the following referred to as a proton pump inhibitor compound, and a pharmaceutical composition comprising a proton pump inhibitor compound. The method and composition are especially aimed for treatment of patients with difficulties to swallow and for pediatric patients. Furthermore, the present invention refers to a method for the manufacturing of said composition and its use in medicine.

### BACKGROUND OF THE INVENTION AND PRIOR ART

Proton pump inhibitor compounds having effect as H⁺K⁺-ATPase inhibitors are for instance compounds known under the generic names omeprazole, lansoprazole, pantoprazole, rabeprazole and esomeprazole.

These active substances are useful for inhibiting gastric acid secretion in mammals and man. In a more general sense, they may be used for prevention and treatment of gastric acid related diseases in mammals and man, including e.g. reflux esophagitis, gastritis, duodenitis, gastric ulcer and duodenal ulcer. Furthermore, they may be used for treatment of other gastrointestinal disorders where gastric acid inhibitory effect is desirable e.g. in patients on NSAID therapy, in patients with Non Ulcer Dyspepsia, in patients with symptomatic gastro-esophageal reflux disease, and in patients with gastrinomas. They may also be used in patients in intensive care situations, in patients with acute upper gastrointestinal bleeding, pre-and postoperatively to prevent acid aspiration of gastric acid and to prevent and treat stress ulceration. Further, they may be useful for prevention and treatment of irritable bowel syndrome (IBS), inflammatory bowel disease (IBD), asthma, laryngitis, Barret's syndrome, sleep apnea, sleep disturbance, psoriasis as well as in the treatment of Helicobacter infections and diseases related to the above.

These active compounds are, however, susceptible to degradation/transformation in acidic and neutral media. The degradation is catalyzed by acidic compounds and is stabilized in mixtures with alkaline compounds. The stability of the active substances is also affected by moisture, heat, organic solvent and to some degree by light.

With respect to the stability properties of the active substances, it is obvious that an oral dosage form should be protected from contact with the acidic gastric juice or comprise suitable components to neutralise the acidic gastric juice so that the active substance can be transferred in intact form to that part of the gastrointestinal tract where pH is near neutral and where rapid absorption can occur.

A pharmaceutical oral dosage form of such an acid labile H⁺K⁺-ATPase inhibitor is best protected from contact with acidic gastric juice by an enteric coating layer. Commonly used solid dosage forms for oral administration are capsules and tablets comprising a multitude of enteric coated pellets of the active ingredient. For instance the following US patents, U.S. Pat. No. 4,853,230, U.S. Pat. No. 4,786,505, U.S. Pat. No. 5,817,338 and U.S. Pat. No. 5,753,265 describe suitable enteric coated preparations. Said preparations contain a core comprising the active ingredient or an alkaline salt thereof, optionally together with an alkaline reacting material, the core is layered with a separating layer and an enteric coating layer. The separating layer may be an optional feature. In order to further enhance the storage stability the prepared formulation may optionally be packed with a desiccant.

However, tablets and capsules are less suitable for administration to patients with difficulties to swallow and for pediatric use. Several of the proton pump inhibitors can be administered orally after dispersion in an aqueous liquid, such as water, fruit juice and fruit sauce. Some of the marked proton pump inhibitors such as omeprazole and lansoprazole, are approved for administration via nasogastric tube, but there is still a need for improvement. For administration via tube, the content of a Prevacid® capsule, i.e. the enteric coated pellets of lansoprazole, will be emptied into 40 mL apple juice and injected through a nasogastric tube. However, only tubes with a relatively large inner diameter, e.g. CH16 (CH=Cherrier), are suitable for this administration.

The tableted dosage form described in U.S. Pat. No. 5,817,338, which is marked outside USA under the trade name Losec® MUPS®, is also suitable for administration via naso-gastric tube after being dispersed in water to a suspension, whereas the product marked in USA under the trade name Prilosec® comprising enteric coated pellets of omeprazole can only be administered through a tube after the content of the capsule has been dispersed in a buffering solution. Further, the product sold under the trade name Prevacid® Oral-suspension should not be given through tube according to the instruction from the manufacture.

Problems that might arise with administration of enteric coated pellets through gastric tube are for instance caused by the size of the enteric coating layered pellets and the inner diameter the tube or the outlet of the syringe, which might cause clogging in the syringe or tube. This is especially critical for pediatric patients where thin tubes are often required. There is also a risk of reduced patient compliance and non-complete dose delivery because of pellets sediment in the glass and/or clogging the syringe used when preparing the suspension. This is especially critical in pediatric use when working with small volumes and doses.

In most cases, parental and /or injectable formulations are not viable alternative because of the need for administration to patients by people trained in medical care and in hospitals.

Therefore, it is still a demand for an improved method for oral administration and for development of new enteric coating layered multiple unit dosage forms that can be used for oral administration. The dosage forms intended for the new administration route should fulfil high demand with respect to chemical and mechanical stability during an extended storage time. Furthermore, there is still a demand for dosage forms having improved patient acceptance, which are easy to handle. One demand on solid dosage forms and compositions is that they should be dispersible in liquids making they possible for oral administration to young children and patients with difficulties to swallow.

### BRIEF DESCRIPTION OF THE INVENTION

One object of the present invention is to provide an improved method for administration via a gastric tube a composition comprising enteric coating layered pellets of a proton pump inhibitor, especially via thin tubes aimed for pediatric use. The expression gastric tube includes naso-gastric tubes as well as other tubes or syringes aimed for feeding a suspension or dispersion to the stomach of a patient.

According to one feature of the invention, the proton pump inhibitor is selected from the group of compounds such as omeprazole, lansoprazole, pantoprazole, rabeprazole or esomeprazole. According to a further feature the compound is esomeprazole prepared for oral administration, preferably as esomeprazole magnesium trihydrate in the form of enteric coating layered pellets. The dosage form comprises the prepared pellets in admixture with a pharmaceutical acceptable thickener. The thickener is capable of forming a viscous medium when dispersed in an aqueous carrier.

Another object of the present invention is to provide a solid composition, which comprises a proton pump inhibitor compound in the form of a multiple of enteric coating layered pellets, the pellets are in mixture with one or more thickeners capable of forming a viscous medium when dispersed in an aqueous carrier. Prior to administration, a ready-to-use composition is prepared by mixing the solid composition of the pharmaceutically active proton pump inhibitor with an aqueous carrier.

It has surprisingly been found that the viscosity of the formed aqueous medium comprising dispersed enteric coating layered pellets of the active substance has an impact on the feeding of the suspension through a tube, such as a gastric or naso-gastric tube. It has been found that the higher viscosity the thinner tubes can be used within certain limits and the solid composition comprising a thickener facilitates and improves the administration, especially to young children. For instance, the handling instruction to patients can be simpler and the administration can be less time consuming.

### DETAILED DESCRIPTION OF THE INVENTION

Active Substance:

Compounds of interest for the improved method and composition of the present invention are compounds of the general formula I or an alkaline salt thereof or one of its single enantiomers or an alkaline salt thereof. 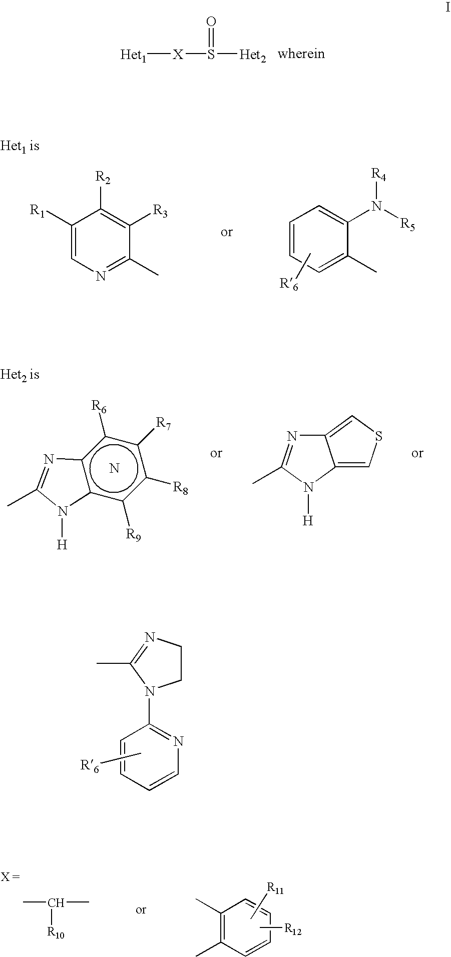
wherein

N in the benzimidazole moiety means that one of the carbon atoms substituted by R₆-R₉ optionally may be exchanged for a nitrogen atom without any substituents;

R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;

R₄ and R₅ are the same or different and selected from hydrogen, alkyl and aralkyl;

R₆′ is hydrogen, halogen, trifluoromethyl, alkyl and alkoxy;

R₆-R₉ are the same or different and selected from hydrogen, alkyl, alkoxy, halogen, halo-alkoxy, alkylcarbonyl, alkoxycarbonyl, oxazolyl, trifluoroalkyl, or adjacent groups R₆-R₉ form ring structures which may be further substituted;

R₁₀ is hydrogen or forms an alkylene chain together with R₃ and

R₁₁ and R₁₂ are the same or different and selected from hydrogen, halogen or alkyl.

In the above definitions alkyl groups, alkoxy groups and moities thereof may be branched or straight C₁-C₉-chains or comprise cyclic alkyl groups, for example cycloalkylalkyl.

Examples of specifically interesting compounds according to formula I are

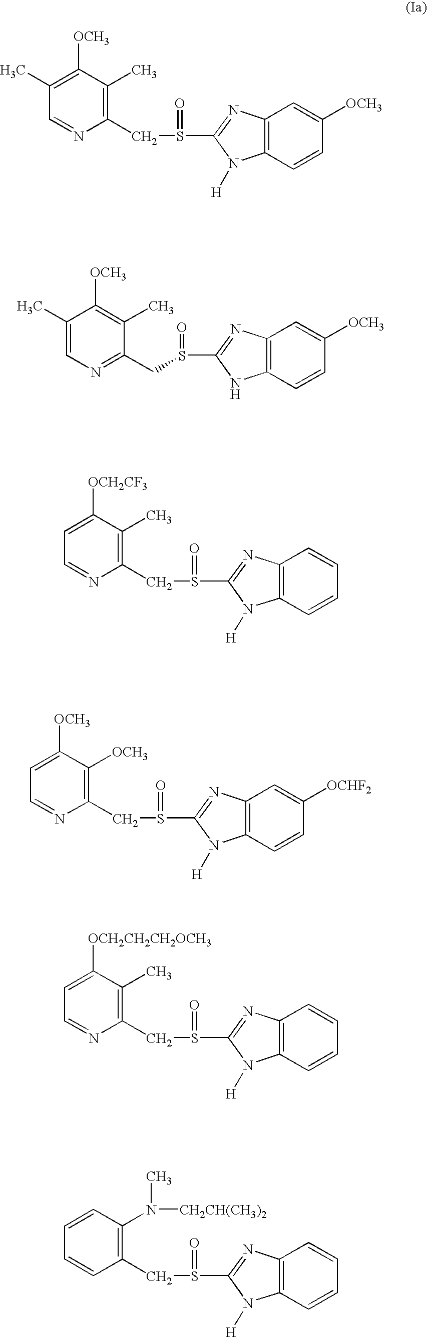

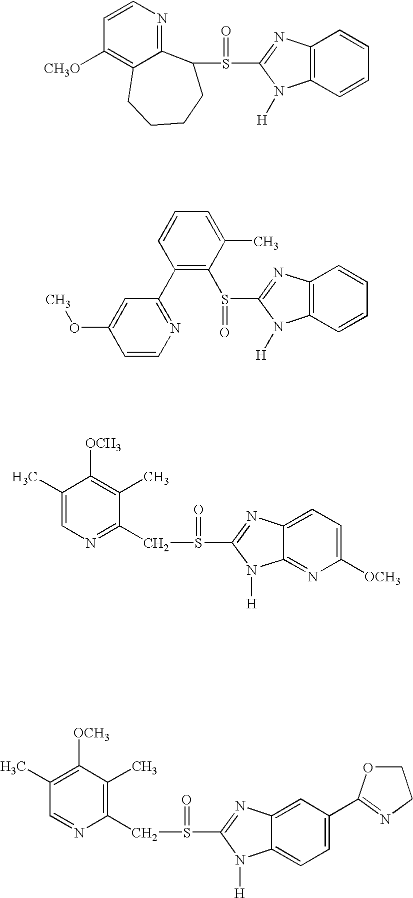

The active compound used in the claimed composition may be used in neutral form or in the form of an alkaline salt, such as for instance the Mg²⁺, Ca²⁺, Na⁺ or K⁺ salts, preferably the Mg²⁺ salts. The compounds may also be used in the form of one of its single enantiomers or alkaline salts thereof, such as exemplified in the second structural formula above.

Preferred proton pump inhibitors for the claimed invention are for instance omeprazole, lansoprazole, pantoprazole, rabeprazole or a pharmaceutical acceptable salt thereof or a single enantiomer thereof, such as esomeprazole magnesium.

Some of the above mentioned compounds are for instance disclosed in EP-A1-0005129, EP-A1-174726, EP-A1-166287, GB 2163747, WO 94/27988, WO95/01977 and WO98/54171 all hereby incorporated by reference.

Enteric Coating Layered Pellets:

Core Material

The core material for the individually enteric coating layered pellets can be constituted according to different principles. Seeds layered with active substance, optionally mixed with alkaline compounds, can be used as the core material for the further processing.

The seeds, which are to be layered with the active substance, can be water insoluble seeds comprising different oxides, celluloses, organic polymers and other materials, alone or in mixtures or water soluble seeds comprising different inorganic salts, sugars, non-pareils and other materials, alone or in mixtures. Further, the seeds may comprise active substance in the form of crystals, agglomerates, compacts etc. According to one aspect of the invention the size of the seeds should be less than approximately 0.8 mm, preferably less than 0.4 mm. The seeds layered with active substance are produced either by powder- or solution/suspension layering using for instance granulating or spray coating/layering equipment.

Before the seeds are layered, the active substance may be mixed with further components. Such components can be binders, surfactants, fillers, disintegrating agents, alkaline additives or other pharmaceutically acceptable ingredients, alone or in mixtures. The binders are for example celluloses such as hydroxypropyl methylcellulose, hydroxypropyl cellulose and carboxymethyl-cellulose sodium, polyvinyl pyrrolidone, sugars, starches and other pharmaceutically acceptable substances with cohesive properties. Suitable surfactants are found in the groups of pharmaceutically acceptable non-ionic or ionic surfactants such as for instance sodium lauryl sulfate.

Alternatively, the H⁺K⁺-ATPase inhibitor or one of its single enantiomers or an alkaline salt thereof, optionally mixed with alkaline compounds and further mixed with suitable constituents can be formulated into core material. Said core materials may be produced by extrusion/spheronization, balling or compression utilizing different equipments. According to one aspect of the invention the size of the formulated core materials is approximately less than 1 mm and preferably in the range between 0.5-1 mm. The manufactured core materials can further be layered with additional ingredients comprising active substance and/or be used for further processing.

The active substance is mixed with pharmaceutical constituents to obtain preferred handling and processing properties and a suitable concentration of active substance in the final mixture. Pharmaceutical constituents such as fillers, binders, lubricants, disintegrating agents, surfactants and other pharmaceutically acceptable additives, can be used.

The active substance may also be mixed with an alkaline pharmaceutically acceptable substance (or substances). Such substances can be chosen among, but are not restricted to, substances such as the sodium, potassium, calcium, magnesium and aluminium salts of phosphoric acid, carbonic acid, citric acid or other suitable weak inorganic or organic acids; aluminium hydroxide/sodium bicarbonate coprecipitate; substances normally used in antacid preparations such as aluminium, calcium and magnesium hydroxides; magnesium oxide or composite substances, such as Al₂O₃.6MgO.CO₂.12H₂O, (Mg₆Al₂(OH)₁₆CO₃.4H₂O), MgO.Al₂O₃.2SiO₂.nH₂O or similar compounds; organic pH-buffering substances such as trihydroxymethylaminomethane, basic amino acids and their salts or other similar, pharmaceutically acceptable pH-buffering substances.

Alternatively, the aforementioned core material can be prepared by using spray drying or spray congealing technique.

Enteric Coating Layer(s)

Before applying enteric coating layer(s) onto the core material in the form of individual pellets, said pellets may optionally be covered with one or more separating layers comprising pharmaceutical excipients optionally including alkaline compounds such as for instance pH-buffering compounds. This/these separating layer(s) separate(s) the core material from the outer layer(s) being enteric coating layer(s).

The separating layer(s) can be applied to the core material by coating or layering procedures in suitable equipments such as coating pan, coating granulator or in a fluidized bed apparatus using water and/or organic solvents for the coating process. As an alternative the separating layer(s) can be applied to the core material by using powder coating technique. The materials for separating layers are pharmaceutically acceptable compounds such as, for instance, sugar, polyethylene glycol, polyvinylpyrrolidone, polyvinyl alcohol, polyvinyl acetate, hydroxypropyl cellulose, methyl-cellulose, ethylcellulose, hydroxypropyl methyl cellulose, carboxymethylcellulose sodium and others, used alone or in mixtures. Additives such as plasticizers, colorants, pigments, fillers, anti-tacking and anti-static agents, such as for instance magnesium stearate, titanium dioxide, talc and other additives may also be included into the separating layer(s).

When the optional separating layer(s) is applied to the core material it may constitute a variable thickness. The maximum thickness of the optional separating layer(s) is normally only limited by processing conditions. The separating layer(s) may serve as a diffusion barrier and may act as a pH-buffering zone. The pH-buffering properties of the separating layer(s) can be further strengthened by introducing into the layer(s) substances chosen from a group of compounds usually used in antacid formulations such as, for instance, magnesium oxide, hydroxide or carbonate, aluminium or calcium hydroxide, carbonate or silicate; composite aluminium/magnesium compounds such as, for instance Al₂O₃.6MgO.CO₂.12H₂O,(Mg₆Al₂(OH)₁₆CO₃.4H₂O), MgO.Al₂O₃.2SiO₂.nH₂O, aluminium hydroxide/sodium bicarbonate coprecipitate or similar compounds; or other pharmaceutically acceptable pH-buffering compounds such as, for instance the sodium, potassium, calcium, magnesium and aluminium salts of phosphoric, carbonic, citric or other suitable, weak, inorganic or organic acids; or suitable organic bases, including basic amino acids and salts thereof Talc or other compounds may be added to increase the thickness of the layer(s) and thereby strengthen the diffusion barrier. The optionally applied separating layer(s) is not essential for the invention. However the separating layer(s) may improve the chemical stability of the active substance and/or the physical properties of the novel multiple unit tableted dosage form.

One or more enteric coating layers are applied onto the core material or onto the core material covered with separating layer(s) by using a suitable coating technique. The enteric coating layer material may be dispersed or dissolved in either water or in suitable organic solvents. As enteric coating layer polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s).

The enteric coating layers contain pharmaceutically acceptable plasticizers to obtain the desired mechanical properties, such as flexibility and hardness of the enteric coating layers. Such plasticizers are for instance, but not restricted to, triacetin, citric acid esters, phthalic acid esters, dibutyl sebacate, cetyl alcohol, polyethylene glycols, polysorbates or other plasticizers.

The amount of plasticizer is optimized for each enteric coating layer formula, in relation to selected enteric coating layer polymer(s), selected plasticizer(s) and the applied amount of said polymer(s), in such a way that the mechanical properties, i.e. flexibility and hardness of the enteric coating layer(s) are adjusted. Additives such as dispersants, colorants, pigments, polymers e.g. poly(ethylacrylat, methylmethacrylat), anti-tacking and anti-foaming agents may also be included into the enteric coating layer(s). Other compounds may be added to increase film thickness and to decrease diffusion of acidic gastric juices into the acidic susceptible material.

To protect an acidic susceptible substance, such as H⁺K⁺-ATPase inhibitors and to obtain an acceptable acid resistance of the multiple unit dosage form according to the invention, the enteric coating layer(s) constitutes a thickness of approximately at least 10 μm, preferably more than 20 μm. The maximum thickness of the applied enteric coating layer(s) is normally only limited by processing conditions.

Over-Coating Layer

Pellets covered with enteric coating layer(s) may further be covered with one or more over-coating layer(s). The over-coating layer(s) can be applied to the enteric coating layered pellets by coating or layering procedures in suitable equipments such as coating pan, coating granulator or in a fluidized bed apparatus using water and/or organic solvents for the layering process. The materials for over-coating layers are pharmaceutically acceptable compounds such as, for instance sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. Additives such as plasticizers, colorants, pigments, fillers, anti-tacking and anti-static agents, such as for instance magnesium stearate, titanium dioxide, talc and other additives may also be included into the over-coating layer(s). Said over-coating layer may further prevent potential agglomeration of the enteric coating layered pellets and protect the enteric coating layer from incompatible excipients or pH values above its dissolution pH, e.g. during the preparation of the suspension, administration and swallowing. The over-coating layer may also include a taste-masking agent. The maximum thickness of the optionally applied over-coating layer(s) is normally only limited by processing conditions.

Pharmaceutically Acceptable Thickener:

Thickeners suitable for the composition of the present invention are thickeners generally used in food industry, such as starch, carrageenan, xanthan gum, guar gum, locust bean gum, tragacanth, gelatin, pectin, modified cellulose derivatives or similar gel forming agents. One single thickener or a combination of thickener can be used.

Alternatively, the enteric coated pellets comprising the pharmaceutically active ingredient may be mixed with a viscous medium such as yoghurt, syrup, sour milk or any aqueous liquid with a similar viscosity. The viscous medium formed or used will provide a homogenous suspension/dispersion to allow the enteric coated pellets floating in the medium.

If no protective over-coating layer is applied on the enteric coating layered pellets the pH value of the viscous medium would be adjusted so that the enteric coating of the pellets is not destroyed during the administration and preparation of the aqueous suspension or dispersion of pellets in the viscous medium, i.e. the enteric coating polymer should not be dissolved in the viscous medium. According to one aspect of the invention, the pH value of the viscous medium is approximately less than 7 and preferably less than 5.6.

The thickener may be mixed with suitable flavour and colour agents and/or sweetening agents acceptable for food products.

It has surprisingly been found that the higher viscosity of the aqueous medium being formed or used for the dispersion/suspension of the enteric coating pellets the narrower gastric tubes can be used.

According to one feature of the invention, the commercial available thickener sold under the trade name “Thick-It” comprising maize starch has shown to be suitable for the improved method for administration via gastric tube. According to another feature, the viscous medium could be yoghurt, syrup or sour milk.

According to one aspect of the invention, the viscosity of the formulation after gelation should be 0.005-10 Pa s and preferably 0.05-5 Pa s, as determined at a shear rate of 10 s⁻¹ from a flow-curve recorded on a rheometer equipped with a plate-plate geometry. Alternatively, the viscosity can be expressed as amount of thickener with respect to amount of aqueous liquid.

Suitable aqueous carriers or liquids to use for administration of the composition through a gastric tube are water, and other pharmaceutically acceptable carriers for oral administration such as fruit juices, dairy products such as milk. Alternatively, the aqueous carrier as such can be used as viscous medium, such as sour milk, yoghurt and liquids with similar viscosity.

The amount of administered aqueous carrier/liquid depends on the amount of active substance, but will generally be in the range of 1-50 mL, preferably 1-30 mL.

Gastric Tubes:

Gastric tubes includes naso-gastric tubes as well as tubes and syringes for feeding. Tubes suitable for the improved method of administration are tubes made of polyvinyl cellulose, polyurethan and similar materials. The size of the tube can vary depending on the patients and the purpose. Adults with swallowing disorders can use tubes with a size measured as CH=Cherrier or “French size” with an inner diameter of approximately CH14-CH20.

Suitable size for pediatric use is approximately a size of CH5-CH10, such as CH5, CH6 and CH8.

Use of Composition.

The composition according to the invention is used for reducing gastric acid secretion. It can be administered one to several times a day. The typical daily dose of the active substance varies and will depend on various factors such as the individual requirements of the patients, the mode of administration and disease. In general the daily dose will be in the range of 1-100 mg of active substance, in some severe cases such as Zollinger-Ellison syndrome there might be a need for higher doses. Preferred doses for adults are 10-80 mg and for pediatric use the preferred doses are 0.5-40 mg of active substance, and for the youngest 0.5- 20 mg depending on the severity.

The present invention is further described and exemplifies in the following experimental report without limiting the invention. The scope of protection is defined by the accompanying claims.

Experimental Report.

Equipment:

Luer lock™ syring: 30 mL.
Tube: Pennine™ health care, Ref No. 15E00; 020E01 (CH 8); 13B01 (CH 10) Flocare™ Pure tube, Ref No. 35242 (CH 6) Argyle Salem Sump™ (CH 10)
Graduated glass: 100 mL
Thickener: Thick-It™
   Preparation of Enteric Coated Pellets:

Esomeprazole magnesium trihydrate was prepared according to U.S. Pat. No. 6,369,085. The prepared active ingredient was formulated into enteric coated pellets as described in U.S. Pat. No. 5,817,338, and the prepared pellets were mixed with the prepared viscous media. The content of U.S. Pat. No. 6,369,085 and U.S. Pat. No. 5,817,338 are hereby incorporated by references.

Preparation of Medium:

Different amounts of Thick-It™ (5, 6, 7 and 8 g, respectively) were mixed with 100 mL tap water, the aqueous solution was mixed vigorously for approx. 1 minute. Prepared enteric coated pellets comprising esomeprazole Mg, corresponding to 10 mg esomeprazole were mixed with 10 mL of the different aqueous, viscous media prepared. The different suspensions were feeded through different tubes.

Pure tap water without thickener was used as reference medium.

The Tubes were Tested According to the Following Procedure:

The tubes were flushed with some water before administration.
1. Remove the piston from a 25-50 mL syringe (Luer-Lock, 30 mL is used) and fill the syringe with approximately 25 mL water.
2. Empty the content of the capsule in the syringe and put the piston back. Leave a space of approximately 5 mL air.
3. Immediately shake the syringe for approximately 15 seconds to disperse the pellets.
4. Hold the syringe with the tip up and check that the tip has not been clogged.
5. Attach the syringe to the tube whilst maintaining the above position (tip pointing up).
6. Shake the syringe and position it with the tip pointing down. Immediately inject 5-10 mL into the tube. Invert the syringe after injection and shake (the syringe must be held with the tip pointing up to avoid clogging of the tip). Turn the syringe with the tip down and immediately inject another 5-10 mL into the tube. Repeat this procedure until the syringe is empty.
7. Fill the syringe with 25 mL of water and 5 mL of air and repeat step 6 if necessary to wash down any sediment left in the syringe.
   Result:

When comparing results from the testing of media with and without thickener, pellets could be administered through more narrow tubes without clogging the tubes when using viscous media for feeding than when using pure tap water. In this experiment, optimal viscosity was obtained when using 6-7 g Thick-It™ in 100 mL tap water.

1. A method for the treatment of gastrointestinal disorders comprising the oral administration of an aqueous suspension via a gastric tube or syringe to a patient in need of such treatment, wherein the aqueous suspension comprises (a) an aqueous carrier and (b) a solid composition comprising a therapeutically effective amount of an acid labile proton pump inhibitor compound in the form of a multiple of enteric coating layered pellets in admixture with at least one pharmaceutically acceptable thickener which forms a viscous medium when dispersed in the aqueous carrier.

2. The method according to claim 1, wherein the thickener is selected from the group consisting of starch, xanthan gum, carrageenan, guar gum, locust bean gum, tragacanth, gelatin, pectin, modified cellulose derivatives and combinations thereof.

3. The method according to claim 1, wherein the thickener is selected from starch and xanthan gum.

4. The method according to claim 1, wherein the solid composition further comprises one or more pharmaceutically acceptable additives selected from the group consisting of flavouring agents, colour agents and sweetening agents.

5. A method for the treatment of gastrointestinal disorders comprising the oral administration of an aqueous suspension via a gastric tube or syringe to a patient in need of such treatment, wherein the aqueous suspension comprises (a) a therapeutically effective amount of an acid labile proton pump inhibitor compound in the form of a multiple of enteric coating layered pellets and (b) a pharmaceutically acceptable viscous aqueous medium and wherein the pellets are dispersed in the viscous aqueous medium to form the aqueous suspension.

6. The method according to claim 5, wherein the viscous aqueous medium is selected from the group consisting of yoghurt, sour milk, and syrup.

7. The method according to claim 5, wherein the viscous aqueous medium is a sugar syrup with a sugar content of at least 63% by weight.

8. The method according to claim 1 or 5, wherein the viscosity of the aqueous suspension is in the range of from 0.005 to 10 Pa s, as determined at a shear rate of 10 s⁻¹ from a flow-curve recorded on a rheometer equipped with a plate-plate geometry.

9. The method according to claim 1 or 5, wherein the viscosity of the aqueous suspension is in the range of from 0.05 to 5 Pa s, as determined at a shear rate of 10 s⁻¹ from a flow-curve recorded on a rheometer equipped with a plate-plate geometry.

10. The method according to claim 1 or 5, wherein the gastric tube has a size in the range of from CH 5 to CH 10 (CH=Cherrier).

11. The method according to claim 1 or 5, wherein the gastric tube has a size in the range of from CH10 to CH20 (CH=Cherrier).

12. The method according to claim 1 or 5, wherein the proton pump inhibitor compound is selected from the group consisting of omeprazole, lansoprazole, pantoprazole, rabeprazole, tenatoprazole, esomeprazole and a pharmaceutically acceptable salt thereof.

13. The method according to claim 1 or 5, wherein the amount of proton pump inhibitor compound that is administered is in the range from 1 to 100 mg.

14. The method according to claim 1, wherein the aqueous carrier is selected from the group consisting of water, fruit juice, syrup and dairy products.

15. The method according to claim 1, wherein the amount of aqueous carrier is in the range from 1 to 35 mL.

16. A solid composition comprising a proton pump inhibitor compound in the form of a multiple of enteric coating layered pellets, wherein the pellets are in admixture with one or more thickeners capable of forming a viscous suspension when dispersed in an aqueous carrier.

17. The composition according to claim 16, wherein the enteric coated pellets are spherical and have a diameter of less than 1 mm.

18. The composition according to claim 16, wherein the enteric coated pellets are spherical and have a diameter of less than 0.5 mm.

19. (canceled)

20. (canceled)
